(19) United States
(12) Patent Application Publication
(10) Pub. No.: US 2025/0064603 A1
(43) Pub. Date: Feb. 27, 2025

(54) EXPANDABLE TRIAL FEMORAL NECK AND ASSOCIATED METHOD OF USE

(71) Applicant: GLOBUS MEDICAL, INC., AUDUBON, PA (US)

(72) Inventors: Edward McGettigan, Jr., Norristown, PA (US); John Loiacono, Collegeville, PA (US); David Stumpo, Trappe, PA (US); Carly McGonagle, Philadelphia, PA (US); Joseph Capozzoli, Mt. Laurel, NJ (US); Yevgeniy Vinshtok, Downingtown, PA (US)

(21) Appl. No.: 18/656,765

(22) Filed: May 7, 2024

Related U.S. Application Data
(63) Continuation-in-part of application No. 18/454,472, filed on Aug. 23, 2023.

(51) Int. Cl. A61F 2/46; A61F 2/36

(52) U.S. Cl. CPC A61F 2/4684; A61F 2/3609; A61F 2002/3625

(57) ABSTRACT

A trial femoral assembly that includes a broach, a neck, and a head. The broach is configured to couple to a femoral shaft. The neck is removably coupled to the broach and includes a center gear configured to rotate about a longitudinal axis of the neck to adjust a length of the neck. The head is removably coupled to the neck and configured for insertion into a hip joint.

### TECHNICAL FIELD

This application is a continuation-in-part of U.S. patent application Ser. No. 18/454,472, filed Aug. 23, 2023. The entire contents of the foregoing disclosure is incorporated by reference herein for all purposes.

### TECHNICAL FIELD

The present disclosure generally relates to an expandible trial femoral neck that allows the surgeon to determine appropriate trial implants (with either a head offset or a stem offset) without dislocating the patient's joint.

### BACKGROUND

In total hip arthroplasty, after broaching and/or reaming the femoral canal and reaming the acetabulum, the surgeon will place different trial components and reduce the joint to find the right range of motion, soft tissue balance, and leg length. The trial components include trial femoral heads and trial femoral necks, and a surgeon will often times trial multiple different offset options to find the best fit for the patient.

Trial heads are offered in sizes 28 millimeters, 32 millimeters, and 36 millimeters. The 28-millimeter heads are offered in offsets of −5 millimeters, −3.5 millimeters, 0 millimeters, +3.5 millimeters, +7 millimeters, +10.5 millimeters, and +12 millimeters. The 32 millimeters and 36 millimeters trial heads are offered in-3.5 millimeters, 0 millimeters, +3.5 millimeters, +7 millimeters, and +10.5 millimeters.

The trial necks are offered in three sizes for use with stems 1/2/3/4, 5/6/7/8, and 9/10/11/12. In addition, standard and lateralized options are available for each size for a total of six trial necks.

Therefore, the total SKUs for just trial heads and trial necks adds up to twenty-three SKUs, taking up a large portion of the graphics cases utilized in surgery. This large number of SKUs typically involves the trial heads being marked or color coded and requires significant space on the instrument tray. This situation also leads to confusion for the scrub technician and surgeon. Thus, there exists a need in the art for an adjustable/expandable trial neck. The trial neck could adjust from standard to lateralized, and/or could expand over a range of trial head sizes.

### SUMMARY

The following objects, features, advantages, aspects, and/or embodiments are not exhaustive and do not limit the overall disclosure. No single embodiment need provide each and every object, feature, or advantage. Any of the objects, features, advantages, aspects, and/or embodiments disclosed herein can be integrated with one another, either in full or in part.

It is a primary object, feature, and/or advantage of the present disclosure to improve on or overcome the deficiencies in the art.

An aspect of the present disclosure is a trial femoral assembly for hip arthroplasty, which includes a head, a neck connected to the head, and a stem or broach connected to the neck, wherein the neck is expandible and collapsible.

Another aspect of the present disclosure is a stem or broach that can move laterally in relation to the longitudinal axis of the neck.

Yet another aspect of the present disclosure is a notched rod located within a housing and within a circular lower neck portion having a tilted spiral cam that interacts with the notched rod to provide movement involving extension or retraction of the neck.

Another feature of the present disclosure is a tilted spiral cam includes a protrusion that engages an opening in the housing to provide a securing mechanism.

Yet another aspect of the present disclosure is a bevel gear located on an outer surface of the head that engages a screw located inside the head to move the neck to provide extension or retraction.

Still, yet another feature of the present disclosure is a driver tool attached to a bevel pinion gear that rotates the bevel gear when the bevel pinion gear is in contact with the bevel gear.

Another feature of the present disclosure is a coupling sleeve having a flange member at an outer end that engages a slot located internally within the head.

Still, another aspect of the present disclosure is a coupling sleeve attached to the neck.

A further feature of the disclosure is a plurality of nested, telescoping threaded elements with oppositely handed threads that extend or retract the neck.

Still, another feature of the present disclosure is a first nested, telescoping threaded element attached to the stem or broach, a third nested, telescoping threaded element attached to the head, and a second nested, telescoping threaded element located between the first nested, telescoping threaded element attached to the stem or broach, and the third nested, telescoping threaded element attached to the head.

Still, yet another feature of the present disclosure is a threaded nut that can travel up and down the first nested, telescoping threaded element and an anti-rotation guide located between the first nested, telescoping threaded element and the third nested, telescoping threaded element to lock rotation of the first nested, telescoping threaded element and the third nested, telescoping threaded element in relationship to each other.

Another aspect of the present disclosure is there is at least a fifty percent overlap between the plurality of nested, telescoping threaded elements at any one time, and the threaded nut has threads in the same direction as the third nested, telescoping threaded element and of an opposite direction to that of the first nested, telescoping threaded element, wherein the third nested, telescoping threaded element is not rotating relative to the first nested, telescoping threaded element so that rotation of the head does not affect an overall length of the trial femoral assembly.

An additional feature of the disclosure is a method of placing a trial femoral assembly on a patient that includes a head, a neck connected to the head, and a stem or broach connected to the neck, where the surgeon can expand or contract the neck to reduce the patient's joint and find the preferred range of motion, soft tissue balance and leg length for the patient.

It is still yet a further object, feature, and/or advantage of the present disclosure is a method of laterally moving the stem or broach in relation to a longitudinal axis of the neck.

Yet another feature of the method of the present disclosure is utilizing a notched rod located within a housing and within a circular lower neck portion having a tilted spiral cam that interacts with the notched rod to provide movement of the neck for extension or retraction.

It is still yet another feature of the method of the present disclosure is utilizing a bevel gear located on the outer surface of the head that engages a screw located inside the head to provide extension or retraction of the neck.

Another feature of the method of the present disclosure is utilizing a driver tool attached to the bevel pinion gear that rotates the bevel gear when the bevel pinion gear is in contact with the bevel gear.

It is yet another feature of the method of the present disclosure is utilizing a plurality of nested, telescoping threaded elements with oppositely handed threads to extend or retract the neck.

It is a further object, feature, and/or advantage of the method of the present disclosure involves utilizing a first nested, telescoping threaded element attached to the stem or broach, a third nested, telescoping threaded element attached to the head, and a second nested, telescoping threaded element located between the first nested, telescoping threaded element and the third nested, utilizing a first nested, telescoping threaded element attached to the stem or broach, a third nested, telescoping threaded element attached to the head, and a second nested, telescoping threaded element located between the first nested, telescoping threaded element and the third nested,

It is still yet a further object, feature, and/or advantage of the method of the present disclosure involving utilizing a threaded nut that can travel up and down the first nested, telescoping threaded element and an anti-rotation guide located between the first nested, telescoping threaded element and the third nested, telescoping threaded element to lock rotation of the first nested, telescoping threaded element and the third nested, telescoping threaded element in relationship to each other.

In another aspect of the present disclosure, a trial femoral assembly is disclosed. The trial femoral assembly includes a broach configured to couple to a femoral shaft, a neck removably coupled to the broach, and a head removably coupled to the neck. The neck includes a center gear configured to rotate about a longitudinal axis of the neck to adjust a length of the neck. Additionally, the head is configured for insertion into a hip joint.

In another aspect of the present disclosure, a neck of a trial femoral assembly is disclosed. The neck includes a base, a trunnion, a pin fixedly coupled to the trunnion, and a center gear. The base is configured to removably couple to a broach of the trial femoral assembly and defines a base cavity therein. The trunnion is configured to removably couple to a head of the trial femoral assembly and defines a trunnion cavity therein. Additionally, the center gear defines a center gear cavity therein, whereby the pin is configured to extend into the center gear cavity and into the base cavity. Moreover, the center gear is configured to rotate about a longitudinal axis of the neck to translate at least one of the center gear and the trunnion along the longitudinal axis.

In another aspect of the present disclosure, a trial femoral system is disclosed. The trial femoral system includes a trial femoral assembly and a driver. The trial femoral assembly includes a broach, a neck, and a head. The broach is configured to be coupled to a femoral shaft. The neck is removably coupled to the broach and includes a center gear configured to rotate about a longitudinal axis of the neck to adjust a length of the neck. Additionally, the head is removably coupled to the neck. The driver is configured to engage the neck to adjust the length of the neck. The drive includes a driveshaft that is configured to rotate about a longitudinal axis of the drive shaft and a mating portion that is configured to engage the center gear and rotate the center gear about the longitudinal axis of the neck based upon rotation of the driveshaft about the longitudinal axis of the driveshaft.

These and/or other objects, features, advantages, aspects, and/or embodiments will become apparent to those skilled in the art after reviewing the following brief and detailed descriptions of the drawings. The present disclosure encompasses (a) combinations of disclosed aspects and/or embodiments and/or (b) reasonable modifications not shown or described.

### BRIEF DESCRIPTION OF THE DRAWINGS

Several embodiments in which the present disclosure can be practiced are illustrated and described in detail, wherein like reference characters represent like components throughout the several views. The drawings are presented for exemplary purposes and may not be to scale unless otherwise indicated.

FIG. 1 is a perspective view of a trial femoral neck from left to right in a first image of a contracted neck position, a second image showing a potential neck expansion, and a third image of an expanded neck position.

FIG. 2 is a perspective view of a trial femoral neck from left to right in a first image of a standard position of the stem in relation to the neck, a second image showing the potential lateral offset of the stem, and a third image of a laterally shifted stem in relation to the neck.

FIG. 3 is a front view of a first embodiment of a neck expansion and contraction mechanism trial femoral neck with a cutaway view within a housing that includes a tilted spiral cam.

FIG. 4 is a side view of a first embodiment of a neck expansion and contraction mechanism trial femoral neck with a cutaway view of a notched rod interacting with the tilted spiral cam shown in FIG. 3.

FIG. 5 is a side view of a first embodiment of a neck expansion and contraction mechanism trial femoral neck rotated ninety degrees from FIG. 4 with a cutaway view of a notched rod interacting with the tilted spiral cam with the notched rod extended and attached to the head and the bottom of the notched rod secured in a circular lower neck enclosure.

FIG. 6 is a perspective view of a second embodiment for a neck expansion and contraction mechanism trial femoral neck that includes a driver tool that rotates a bevel pinion gear that engages a bevel gear on a head that expands or contracts the length of the neck.

FIG. 7 is a perspective cutaway view of a second embodiment for a neck expansion and contraction mechanism trial femoral neck that includes a driver tool that rotates a bevel pinion gear that engages a bevel gear on a head that expands or contracts a length of a neck by engaging a screw located within a coupling sleeve that is secured within a head.

FIG. 8 is a cutaway perspective view of a third embodiment for a neck expansion and contraction mechanism trial femoral neck that includes a first nested, telescoping threaded element attached to the stem, a third nested, telescoping threaded element attached to the head, and a second nested, telescoping threaded element located between the first nested, telescoping threaded element and the third nested, telescoping threaded element with a threaded nut that can travel up and down the second nested, telescoping threaded element and an anti-rotation guide located between the second nested, telescoping threaded element and the third nested, telescoping threaded element to lock rotation of the second nested, telescoping threaded element and the third nested, telescoping threaded element in relationship to each other in a contracted position.

FIG. 9 is a cutaway perspective view of a third embodiment for a neck expansion and contraction mechanism trial femoral neck shown in FIG. 8 that includes a first nested, telescoping threaded element attached to the stem; a third nested, telescoping threaded element attached to the head, and a second nested, telescoping threaded element located between the first nested, telescoping threaded element and the third nested, telescoping threaded element with a threaded nut that can travel up and down the second nested, telescoping threaded element and an anti-rotation guide located between the second nested, telescoping threaded element and the third nested, telescoping threaded element to lock rotation of the second nested, telescoping threaded element and the third nested, telescoping threaded element in relationship to each other in an expanded position.

FIG. 10 is a perspective view of a third embodiment for a neck expansion and contraction mechanism trial femoral neck shown in FIG. 8 that includes a first nested, telescoping threaded element attached to the stem; a third nested, telescoping threaded element attached to the head, and a second nested, telescoping threaded element located between the first nested, telescoping threaded element and the third nested, telescoping threaded element with a threaded nut that can travel up and down the second nested, telescoping threaded element and an anti-rotation guide located between the second nested, telescoping threaded element and the third nested, telescoping threaded element to lock rotation of the second nested, telescoping threaded element and the third nested, telescoping threaded element in relationship to each other in an expanded position.

FIG. 11 is a perspective view of a trial femoral assembly in accordance with the present teachings.

FIG. 12A is a perspective view of a neck of a trial femoral assembly in a retracted position.

FIG. 12B is a cross-sectional view of the neck of FIG. 12A.

FIG. 13A is a perspective view of the neck of FIG. 12A in an extended position.

FIG. 13B is a cross-sectional view of the neck of FIG. 13A.

FIG. 14A is a perspective view of a driver that is configured to drive extension and retraction of the neck of FIGS. 12A-13B.

FIG. 14B is a cross-sectional view of the driver of FIG. 14A.

FIG. 15 is a close-up view of a mating portion of the driver of FIG. 14B.

An artisan of ordinary skill in the art need not view, within isolated figure(s), the near-infinite distinct combinations of features described in the following detailed description to facilitate an understanding of the present disclosure.

### DETAILED DESCRIPTION

The present disclosure is not to be limited to that described herein. Mechanical, electrical, chemical, procedural, and/or other changes can be made without departing from the spirit and scope of the present disclosure. Unless otherwise indicated, no features shown or described are essential to permit basic operation of the present disclosure.

Trial femoral neck segments and trial femoral modular heads are utilized to assess proper component position, joint stability, range of motion, and leg length.

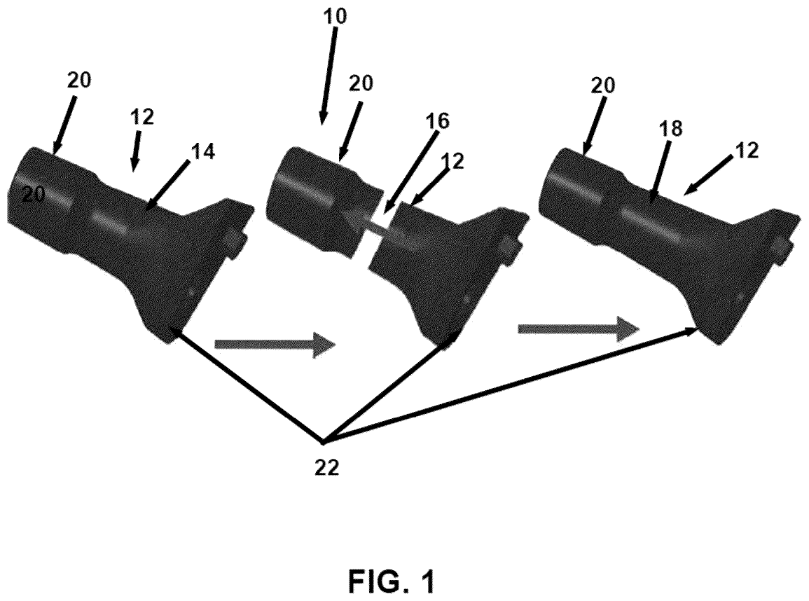

Referring now to FIG. 1, a trial femoral head and neck assembly is generally indicated by the numeral 10. The adjustable femoral neck portion 12 of the trial femoral head and neck assembly 10 can adjust from standard to lateralized, and/or could expand over a range of sizes of trunnion 20. Moreover, the adjustable femoral neck portion 12 can expand over a range of sizes that allows the surgeon to adjust the adjustable femoral neck portion 12 while just using one neutral (+0 millimeters) trial head and would allow the surgeon to easily change the offset while the patient's joint is still reduced. The expansion mechanism of the adjustable femoral neck portion 12 will be on the internal portion of the adjustable femoral neck portion 12. It will be controlled by a separate instrument that the surgeon could tum with a click, indicating that it has adjusted to a new size. The trunnion 20 sizes range from −5 millimeters to +12 millimeters, so ideally, the adjustable femoral neck portion 12 will be able to expand throughout that entire range. There is a stem 22 for attachment to the femur. There can be a broach representative of the final stem implant geometry substituted for the stem 22. The is a first length 14 of the adjustable femoral neck portion 12, a visual depiction of the expansive of neck portion along the neck axis 16 of the adjustable femoral neck portion 12, and a second length 18 of the adjustable femoral neck portion 12. The images in FIG. 1 show how the adjustable femoral neck portion 12 will expand along the axis of the neck to lengthen. It will expand in increments so that the user can easily tell how much they are expanding. Preferably, but not necessarily, it will also be laser marked to show the exact dimension.

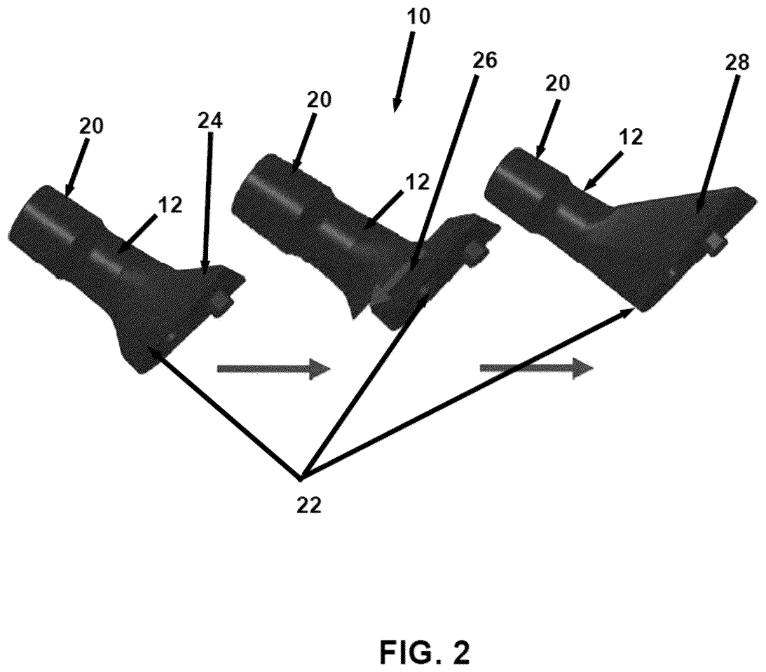

Referring now to FIG. 2, another feature is to make the trial femoral head and neck assembly 10 adjustable so that a change from standard to lateralized can be accomplished. Currently, there needs to be six total trial necks due to there being three different neck lengths, and one standard and one lateralized option for each one. If the adjustable femoral neck portion 12 can be adjusted to be lateralized, that would reduce the total number of trial necks from six to three SKUs. As shown, stem 22 can go from a standard position of the stem 24 in relation to the adjustable femoral neck portion 12, as shown by a lateral change of stem 26 in relation to the adjustable femoral neck portion 12 to a lateralized position of the stem 22 to the adjustable femoral neck portion 28. The trunnion 20 can be moved back and forth and could click into the correct place so that the user obtains tactile feedback to verify that the adjustable femoral neck portion 12 is located in the proper position. Preferably, there would be an indication showing the level of offset.

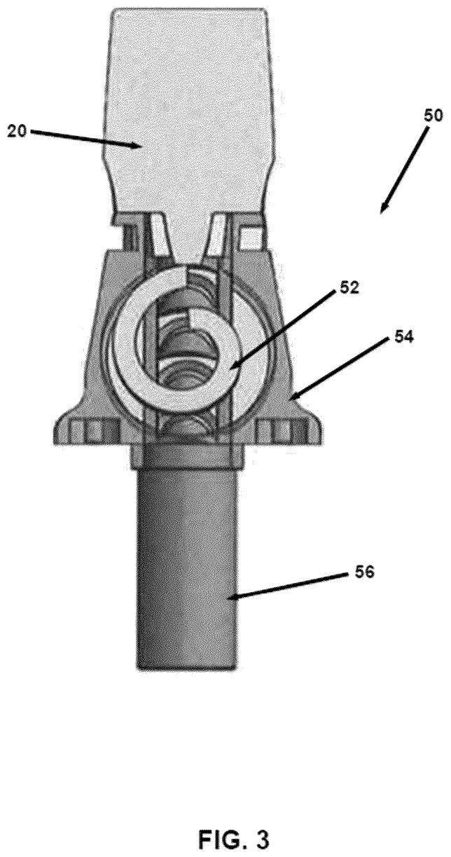

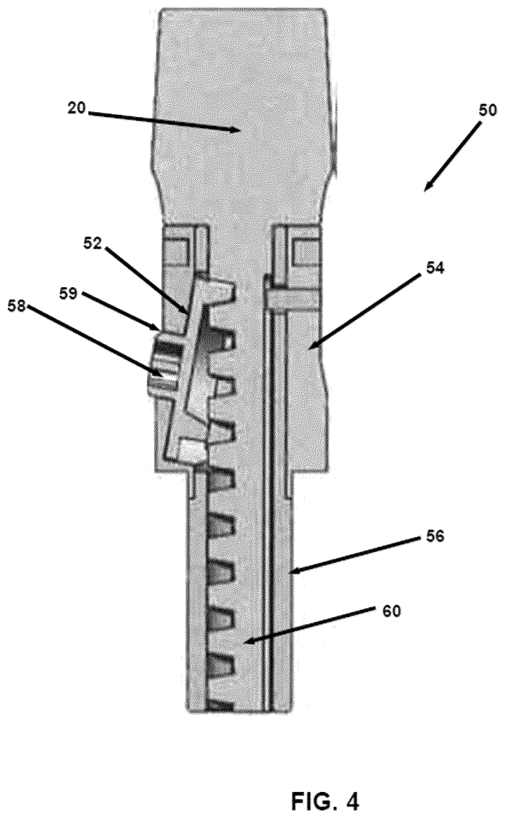

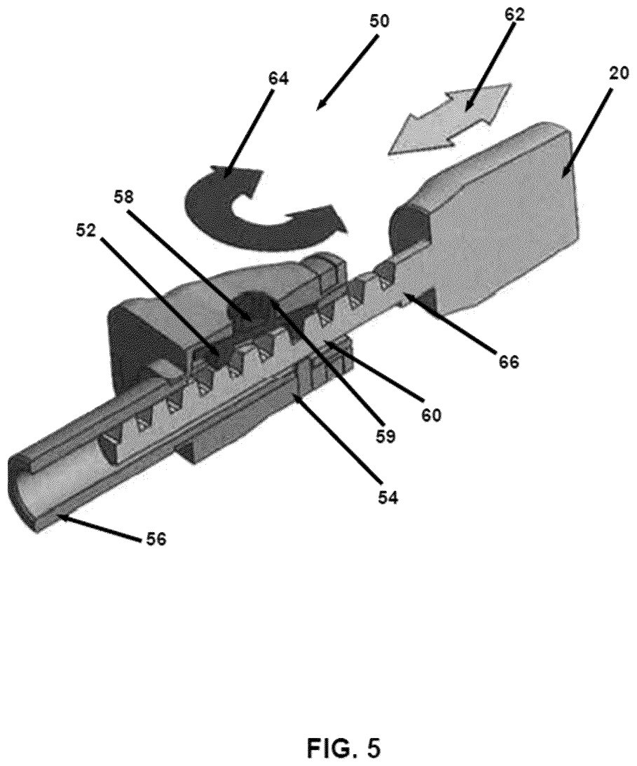

There is a first embodiment of a neck lengthening device that is generally indicated by the numeral 50 in FIGS. 3, 4, and 5. Positioned below the trunnion 20 is notched rod 60 with surfaces that connect to surfaces of a tilted spiral cam 52. The tilted spiral cam 52 is located within housing 54. This neck lengthening device 50 provides an extension of the adjustable femoral neck portion 12, shown in FIG. 5, that cannot be back driven by pressure from the tissue or rotation of the trunnion 20. The amount of extension 66 of the trunnion 20 can be accurately controlled or precisely “dialed in” based on the dimensions of the tilted spiral cam 52. The tilted spiral cam 52 includes a protrusion 58 that engages an opening 59 in the housing 54 to provide a securing mechanism. The longitudinal neck movement is generally indicated by the numeral 62, and the circular spiral cam movement that is converted to longitudinal neck movement is indicated by the numeral 64. Housing 54 is positioned above the neck portion 56, which is cylindrical and encloses the notched rod 60.

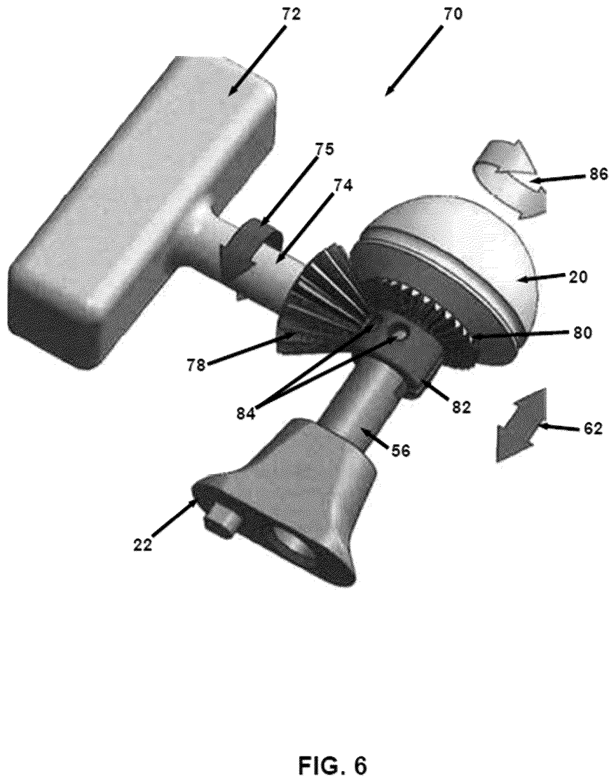

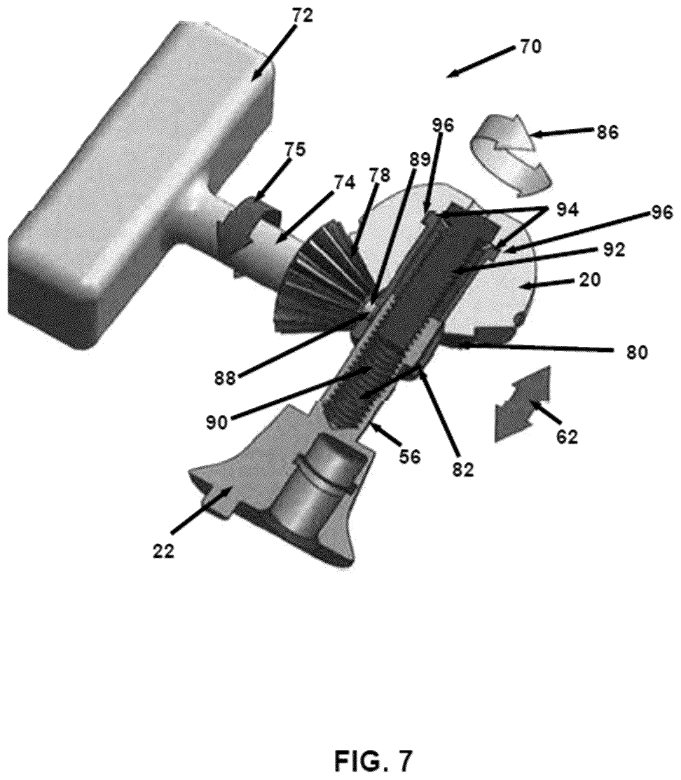

A second embodiment of a neck lengthening device is generally indicated by the numeral 70 in FIGS. 6 and 7. There is a driver tool 72 that provides a rotational force. Numerous embodiments of various types of tools can provide this type of rotational force comparable to a hand drill. The driver tool 72 includes an enclosed rotating shaft 74 that is attached to a bevel pinion gear 78. The direction of rotation of the rotating shaft 74 is indicated by the numeral 75. The bevel pinion gear 78 engages a bevel gear 80 that is attached to the trunnion 20 with the rotation indicated by the numeral 86. As before, the neck portion 56 is positioned on top of the stem 22.

As shown in FIG. 7, there is a coupling sleeve 82 that includes a bushing 88 that retains a circular protrusion 89 associated with the bevel pinion gear 78. There is retained flange 94 as an upper component of the coupling sleeve 82 that is held by a slot 96 in the trunnion 20. The bevel gear 80 engages a screw 92, rotatable in a threaded internal passage 90, that provides the power to extend and retract the trunnion 20. The bevel gear 80 is also connected to the coupling sleeve 82, which serves to provide a proper alignment of the bevel pinion gear 78 and bevel gear 80 for engagement, as well as provide a bearing surface to guide the extension and retraction of neck portion 56 indicated by numeral 62. The coupling sleeve 82 is connected to the neck portion 56 by connecting screws 84, as shown in FIG. 6.

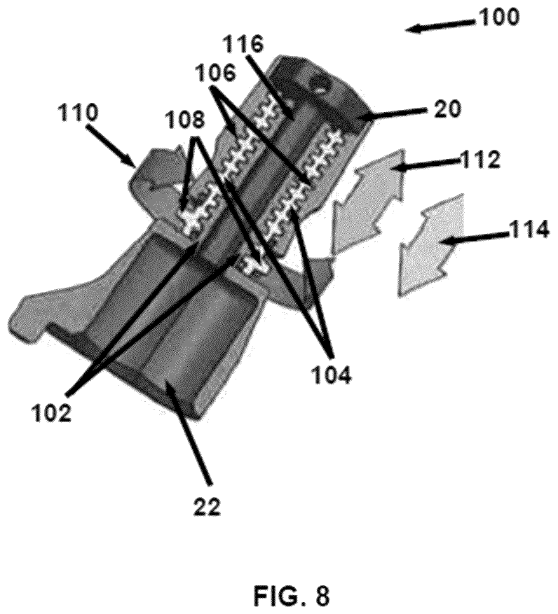

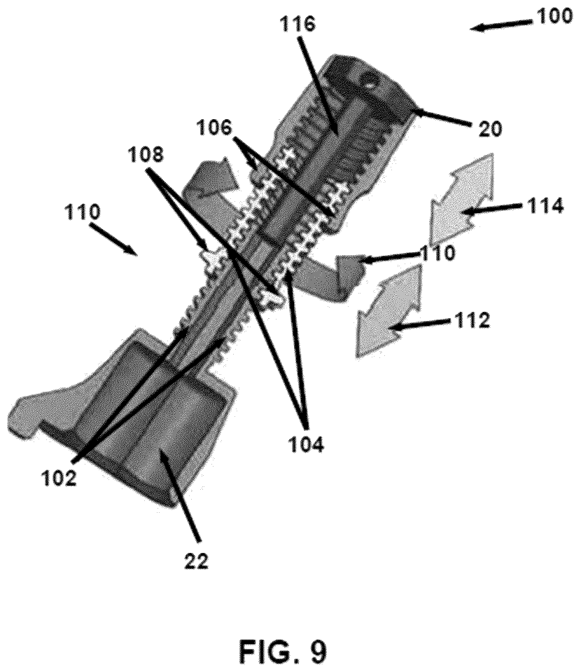

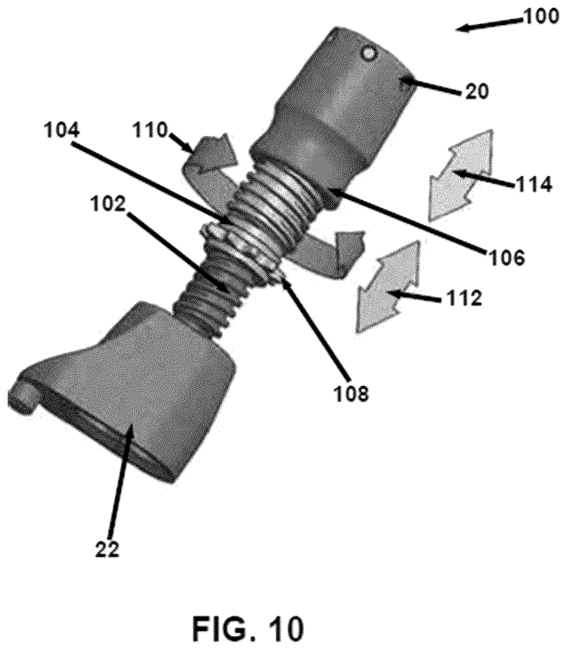

A third embodiment of a neck lengthening device is generally indicated by the numeral 100 in FIGS. 8, 9, and 10. This neck lengthening device 100 includes nested, telescoping, threaded elements, e.g., three, with oppositely handed threads. In an illustrative, but nonlimiting, embodiment, a first nested, telescoping threaded element 102, e.g., right-hand screw, has a thread running in one direction, e.g., clockwise, with a nut 108 with matching internal thread that can go up and down the first nested, telescoping threaded element 102.

This nut 108 has an external thread of opposite handedness, e.g., counterclockwise, which engages a third nested, telescoping threaded element 106, preferably with a matching internal thread, e.g., counterclockwise. This third nested, telescoping threaded element 106 is connected to the first nested, telescoping threaded element 102 via an anti-rotation guide 116, which locks the rotation of the first nested, telescoping threaded element 102 and third nested, telescoping threaded element 106. When the second nested and telescoping threaded element 104 is rotated, it rides up the first nested, telescoping threaded element 102 and forces the third nested, telescoping threaded element 106 to extend in the same direction. Because all telescoping threaded elements 102, 104, and 106 are nested inside each other and are “telescoping,” they maintain overlap from 50% to 100% at any one time, providing bending rigidity or this extending structure. Rotational movement is generally indicated by the numeral 110 of the telescoping threaded elements 102, 104, and 106.

Also, because the third nested, telescoping threaded element 106 is not rotating relative to the first nested, telescoping threaded element 102 that forms a base element, the rotation of the trunnion 20 does not affect the overall length of the neck lengthening device 100. The longitudinal extension and contraction of the second nested and telescoping threaded element 104 is indicated by numeral 112, and the longitudinal extension and contraction of the third nested, and telescoping threaded element 106 is indicated by numeral 114.

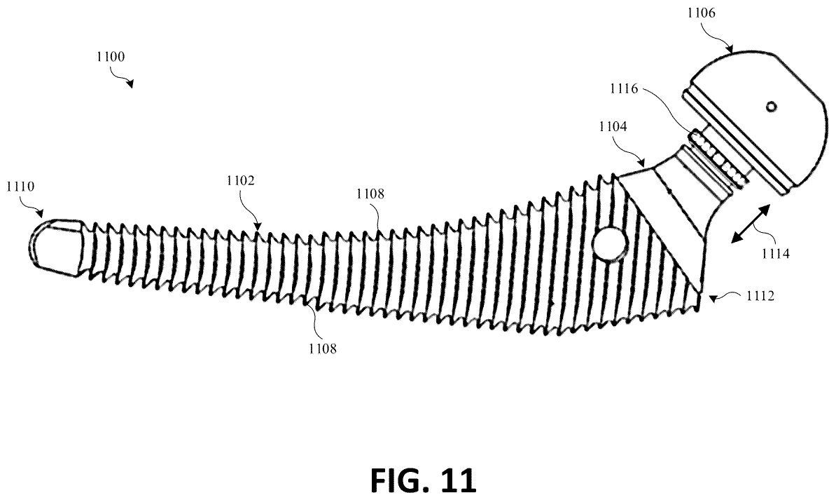

FIG. 11 illustrates a trial femoral assembly 1100 in accordance with the present teachings. The trial femoral assembly 1100 may be similar to or include the trial femoral head and neck assembly 10 discussed above.

As discussed above, the trial femoral assembly 1100 may provide a surgeon with an alternative means to determine the best shape and/or fit for a potential implant. That is, the trial femoral assembly 1100 may provide flexibility and/or adjustability to determine a proper range of motion, soft tissue balance, leg length, other characteristics of the potential implant, or a combination thereof. That is, the trial femoral assembly 1100 may provide the surgeon a self-contained and adjustable trial sample that may eliminate the need for myriad trial components conventionally used. By way of example, a conventional trial may require the surgeon to fit, interchange, and adjust various components having different geometries (e.g., various necks and/or heads). However, the trial femoral assembly 1100 may be adjustable to eliminate some or all of the trial components conventionally used.

Referring now more specifically to FIG. 11, the trial femoral assembly 1100 may include a broach 1102, a neck 1104, and a head 1106. The broach 1102 may be configured to be coupled to a femoral shaft of a person (e.g., a patient) or any mammal (humans, primates, quadruped animals, etc.) By way of example, the broach 1102 may include one or more ribs 1108 extending along all or a portion of the broach 1102 to engage a femoral canal of the femoral shaft. For example, a distal end 1110 of the broach 1102 may be inserted into (e.g., impacted into) the femoral canal such that the ribs 1108 may engage an interior surface of the femoral canal and maintain coupling between the broach 1102 and the femoral shaft (e.g., coupling between the trial femoral assembly 1100 and the femoral shaft).

The neck 1104 may be removably coupled to the broach 1102. For example, the neck 1104 may be removably coupled to a proximal end 1112 of the broach 1102 that is located on an opposite end of the broach 1102 from the distal end 1110. That is, the proximal end 1112 and the distal end 1110 may be opposing ends of the broach 1102 with respect to a length of the broach 1102. Based on such structure, the broach 1102 may be coupled to the femoral shaft as discussed above prior to, or after, coupling the neck 1104 to the broach 1102. For example, the broach 1102 may be initially impacted into the femoral canal and the neck 1104 may thereafter be coupled to the broach at or near the proximal end 1112 of the broach 1102. However, the above is not intended to limit the positioning of the neck 1104 and the broach 1102 with respect to one another, and any manner of removable and/or fixed coupling may be possible.

The head 1106 of the trial femoral assembly 1100 may be removably coupled to the neck 1104 and configured to be inserted into a hip joint of the person (e.g., the patient). As a result, the trial femoral assembly 1100 may mimic a potential implant that may connect the femoral shaft of the person to the hip. To further resemble the potential implant, the trial femoral assembly 1100 may include one or more movable components to adjust an overall size and/or shape of the trial femoral assembly 1100.

By way of example, the neck 1104 may include a center gear 1116 that may be configured to rotate to adjust a length of the neck 1104. As discussed further below, the center gear 1116 may rotate to increase and/or decrease a distance between the proximal end 1112 of the broach 1102 and the head 1106. That is, all or a portion of the broach 1102, all or a portion of the neck 1104, all or a portion of the head 1106, or a combination thereof may move in the direction 1114 based upon rotation of the center gear 1116 to adjust the length of the neck 1104. As a result, an overall size (e.g., length and/or offset) of the trial femoral assembly 1100 may be increased and/or decreased.

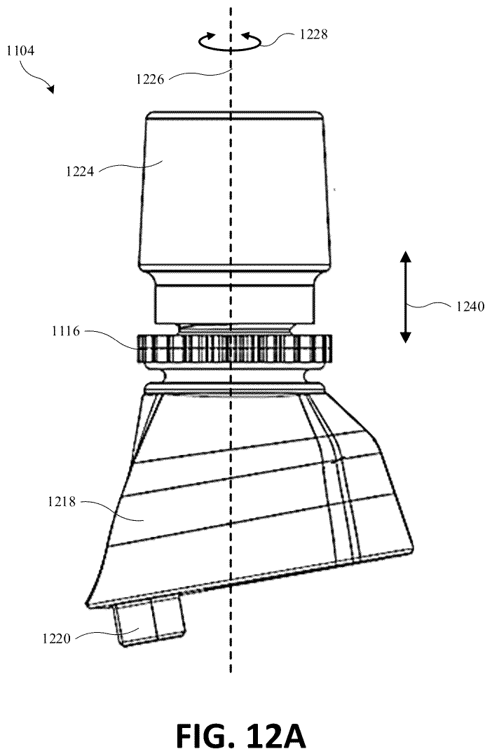

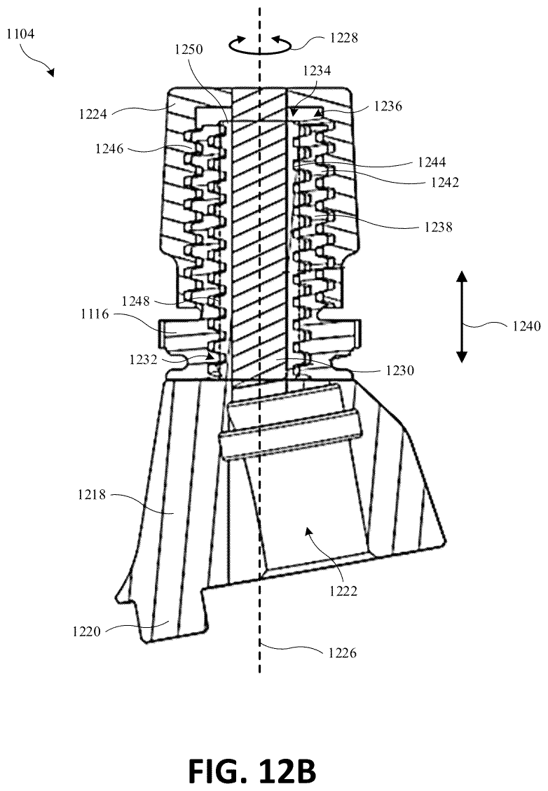

FIG. 12A illustrates a perspective view of the neck 1104 of the trial femoral assembly 1100. FIG. 12B is a cross-sectional view of the neck 1104 shown in FIG. 12A. The neck 1104 is shown in a retracted (e.g., non-extended) position.

As discussed above, the neck 1104 may be removably coupled to the broach 1102 shown in FIG. 11. For example, the neck 1104 may include a base 1218 that removably couples to the broach 1102. The base 1218 may include one or more mating and/or engaging features that are configured to interact (e.g., interconnect) with one or more respective mating and/or engaging features of the broach 1102. By way of example, the base 1218 may include a projection 1220 that may extend from the base 1218. The projection 1220 may extend towards the broach 1102 such that a portion of the broach 1102 (e.g., the proximal end 1112 of the broach 1102) may receive the projection 1220 to align and/or couple the neck 1104 to the broach 1102. The broach 1102 may define a cavity or include another type of receiving feature that receives all or a portion of the projection 1220. As such, the cavity or receiving feature of the broach 1102 may be complementary in shape to the projection 1220.

The base 1218 may include one or more additional, or alternative, mating and/or engaging features other than the projection 1220. For example, the base 1218 may also define a receiving portion 1222 therein. The receiving portion 1222 may be configured to receive a projection of the broach 1102 to removably connect the neck 1104 to the broach 1102. The projection of the broach 1102 may be similar to the projection 1220 or may have a different overall configuration. For example, the projection of the broach 1102 may include one or more mechanical interlocking features (e.g., threading, fingers, pins, etc.) that may be received by the receiving portion 1222 to removably couple the neck 1104 to the broach 1102. Thus, the base 1218 may facilitate easy connection and disconnection between the broach 1102 and the neck 1104.

The neck 1104 may also include a trunnion 1224. The trunnion 1224 may be configured to removably couple to the head 1106 of the trial femoral assembly 1100. For example, the trunnion 1224 may be received by a socket or other receiving feature of the head 1106 to removably couple the head 1106 to the trunnion 1224. As a result, neck 1104 and the head 1106 may be easily connected and disconnected from one another.

It should be noted that while connection of the neck 1104 to the broach 1102 and the head 1106 are discussed in detail herein, the neck 1104 may also be configured to removably couple to a variety of other broaches and/or heads. That is, the neck 1104 may provide a universal or common component that may be used with interchangeable broaches and/or heads, thereby providing further customization and functionality of the trial femoral assembly 1100.

Turning back to FIGS. 12A and 12B, the neck 1104 may also include the center gear 1116 as discussed above. The center gear 1116 may be positioned between the base 1218 and the trunnion 1224. The center gear 1116 may be movably and/or fixedly coupled to one or more portions of the neck 1104. For example, the center gear 1116 may be movably coupled to the trunnion 1224, either directly or indirectly. Based on such positioning, the center gear 1116 may be configured to rotate about a longitudinal axis 1226 of the neck 1104 to adjust a length of the neck 1104. By way of example, the center gear 1116 may be positioned between the base 1218 and the trunnion 1224 along the longitudinal axis 1226. The center gear 1116 may be configured to rotate about the longitudinal axis 1226 in a direction 1228 to adjust the length of the neck 1104 such that a distance between the broach 1102 and the head 1106 of the trial femoral assembly 1100 is increased or decreased.

To facilitate such adjustability, the neck 1104 may further include a pin 1230 fixedly coupled to the trunnion 1224 of the neck 1104. The pin 1230 may extend between the trunnion 1224 and the base 1218 to maintain a rotational position between the trunnion 1224 and the base 1218 with respect to the longitudinal axis 1226. The base 1218 and the trunnion 1224 may also be movably connected via the pin 1230 to facilitate translational movement of the trunnion 1224 towards and away from the base 1218.

By way of example, the center gear 1116 may define a cavity 1232 therein. Similarly, the base 1218 may define a cavity 1234 therein. The pin 1230 may be configured to extend into or through the cavity 1232 of the center gear 1116 and into the cavity 1234 of the base 1218. As a result, the pin 1230 may movably couple the center gear 1116 and the trunnion 1224 to the base 1218 while maintaining a rotational orientation between the base 1218 and the trunnion 1224.

The center gear 1116 may also at least partially extend into a cavity 1236 defined by the trunnion 1224 to maintain engagement between the trunnion 1224, the center gear 1116, and the pin 1230 (e.g., the base 1218). For example, the center gear 1116 may include a cylindrical body 1238 that is at least partially inserted into the cavity 1236 of the trunnion 1224 to maintain engagement between the trunnion 1224 and the center gear 1116.

As discussed above, the center gear 1116 may be configured to rotate about the longitudinal axis 1226 of the neck 1104 to adjust a length of the neck 1104. In particular, rotation of the center gear 1116 in the direction 1228 about the longitudinal axis 1226 increases and/or decreases a distance between the trunnion 1224 and the base 1218. That is, the center gear 1116 is configured to rotate about the longitudinal axis 1226 to translate the center gear 1116, the trunnion 1224, or both in a direction 1240 towards and/or away from the base 1218 when the base 1218 remains stationary. However, in situations where the center gear 1116 remains translationally fixed and is rotated in the direction 1228, the base 1218 and the trunnion 1224 may translate in the direction 1240.

By way of example, the center gear 1116 and/or the trunnion 1224 may be configured to translate in the direction 1240 along the longitudinal axis 1226 based upon rotation of the center gear 1116 in the direction 1228 about the longitudinal axis 1226. As a result, a distance between the base 1218 and the trunnion 1224 may be adjusted (e.g., increased or decreased), which may in turn adjust (e.g., increase or decrease) a distance between the broach 1102 and the head 1106.

As mentioned above, the neck 1104 of FIGS. 12A and 12B is shown in a retracted position. The retracted or non-extended position may be a position of the trunnion 1224 and the center gear 1116 such that the distance between the trunnion 1224 and the base 1218 is at a minimum. However, it should be noted that the non-extended position may be a neutral position somewhere between the minimum and maximum distances possible between the trunnion 1224 and the base 1218. For example, from the retracted position, the center gear 1116 may be rotated to adjust the distance between the trunnion 1224 and the base 1218 in a range of about −3.5 mm to about 10.5 mm. However, any range may be possible.

The center gear 1116 may be rotated about the longitudinal axis 1226 to translate the center gear 1116 and/or the trunnion 1224 in the direction 1240. To facilitate such translation, the center gear 1116 may include external threading 1242 and internal threading 1244. The external threading 1242 may engage threading 1246 of the trunnion 1224 and the internal threading 1244 may engage threading 1248 of the base 1218 such that rotation of the center gear 1116 may translate the center gear 1116—and thus the trunnion 1224—in the direction 1240 along a cylindrical body 1250 of the base 1218 (e.g., a portion of the base 1218 projecting from the base 1218 that includes the threading 1246). Similarly, rotation of the center gear 1116 may also translate the trunnion 1224 in the direction 1240 along the center gear 1116.

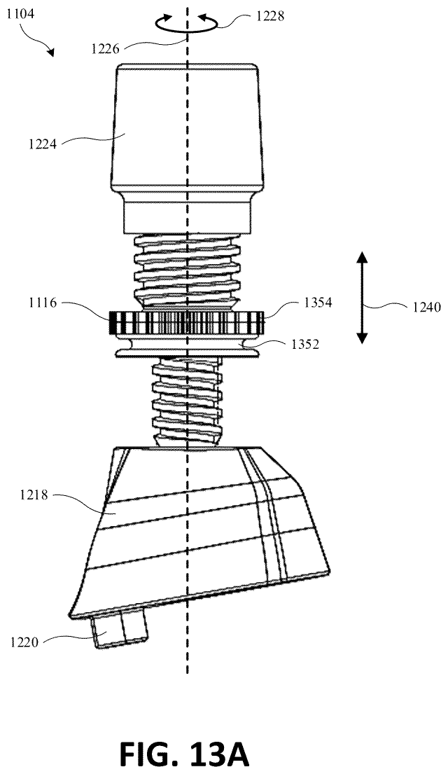

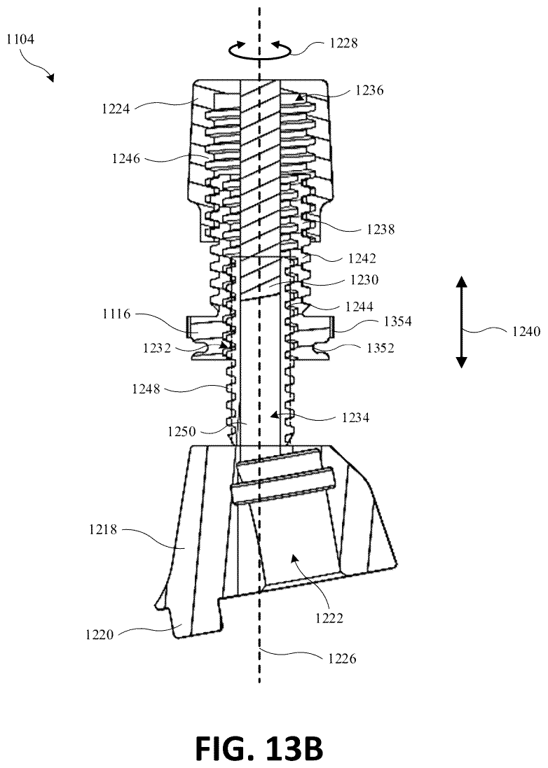

To further illustrate the above movement, FIG. 13A illustrates a perspective view of the neck 1104. FIG. 13B is a cross-sectional view of the neck 1104 shown in FIG. 13A. While FIGS. 12A and 12B illustrate the neck 1104 in a retracted (e.g., non-extended) position, the FIGS. 13A and 13B illustrate the neck 1104 in an extend position. As discussed above, threaded engagement between the base 1218, the center gear 1116, and the trunnion 1224 may facilitate translation of the of the center gear 1116 and/or the trunnion 1224 in the direction 1240.

To better illustrate articulation of the neck 1104, an example operation to adjust a length of the neck 1104 will be discussed in further detail with respect to FIGS. 12A-13B. To initiate operation (e.g., extension or retraction) of the neck 1104, the center gear 1116 may be rotated about the longitudinal axis 1226 in the direction 1228. As discussed further below, a secondary tool, such as a driver, may be removably coupled to the center gear 1116 via a groove 1352 of the center gear 1116 such that the secondary tool (e.g., the driver) may engage teeth 1354 of the center gear 116 to drive rotation of the center gear 1116 in the direction 1228.

As the center gear 1116 rotates in the direction 1228, the external threading 1242 of the center gear 1116 engages the threading 1246 of the trunnion 1224 (e.g., the threading 1246 located in the cavity 1236 of the trunnion 1224) such that the rotation of the center gear 116 translates the trunnion 1224 along the longitudinal axis 1226 in the direction 1240 to adjust the length of the neck 1104. It should be noted that one or more end stops may exist on the center gear 1116 and/or the trunnion 1224 to prevent overtravel of the trunnion 1224 and/or disconnection between the trunnion 1224 and the center gear 1116.

Similarly, as the center gear 1116 rotates in the direction 1228, the internal threading 1244 of the center gear 1116 located within the cavity 1232 of the center gear 1116 engages the threading 1248 of the base 1218 that is located on the cylindrical body 1250 of the base 1218 such that the rotation of the center gear 1116 in the direction 1228 translates the center gear 1116 along the longitudinal axis 1226 in the direction 1240 to further adjust the length of the neck 1104. The center gear 1116 and/or the base 1218 may include one or more end stops to prevent overtravel of the center gear 1116 and/or disconnection between the center gear 1116 and the base 1218.

Additionally, as discussed above, the neck 1104 may maintain rotational orientation of the trunnion 1224 and the base 1218 with respect to one another and with respect to the longitudinal axis 1226. In particular, the pin 1230 may be located within the cavity 1236 of the trunnion 1224 and may extend into the cavity 1234 of the base 1218 (e.g., the cavity 1234 located in the cylindrical body 1250 of the base 1218). The pin 1230 and the cavity 1234 of the base 1218 may create an anti-rotation coupling such that the trunnion 1224 and the base 1218 are unable to rotate with respect to one another. Moreover, the pin 1230 may be guided to move within the cavity 1234 of the base 1218 during translation of the trunnion 1224 in the direction 1240. Thus, the pin 1230 may extend along the longitudinal axis 1226 such that the pin 1230, the base 1218, and the center gear 1116 are coaxial along the longitudinal axis 1226.

It should also be noted that threading pitch and/or orientation of any of the threading discussed above is not particularly limited. For example, all threading may be of an equal pitch to ensure equal translation of the trunnion 1224 and the center gear 1116 with respect to one another and with respect to the base 1218. However, threading between components may also have different pitches. Similarly, orientation between threading may be similar or may vary. For example, the threading 1246 of the trunnion 1224 and the external threading 1242 of the center gear 1116 may be oriented in a similar manner (e.g., right-handed threading) while the internal threading 1244 of the center gear 1116 and the threading 1248 of the base 1218 may be oriented in a similar manner (e.g., left-handed threading) that opposes the threading 1246 of the trunnion 1224 and the external threading 1242 of the center gear 1116. As such, rotation of the center gear 1116 may drive movement of all components working together to adjust the length of the neck 1104.

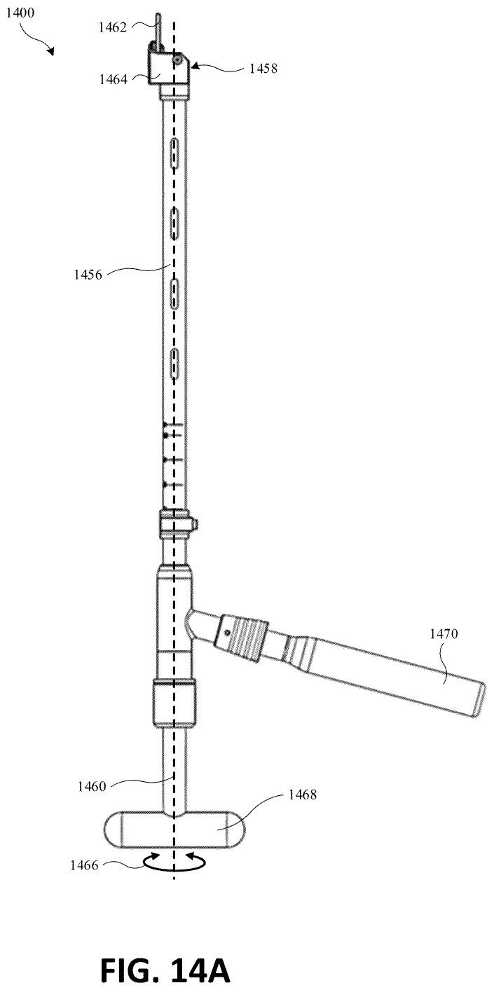

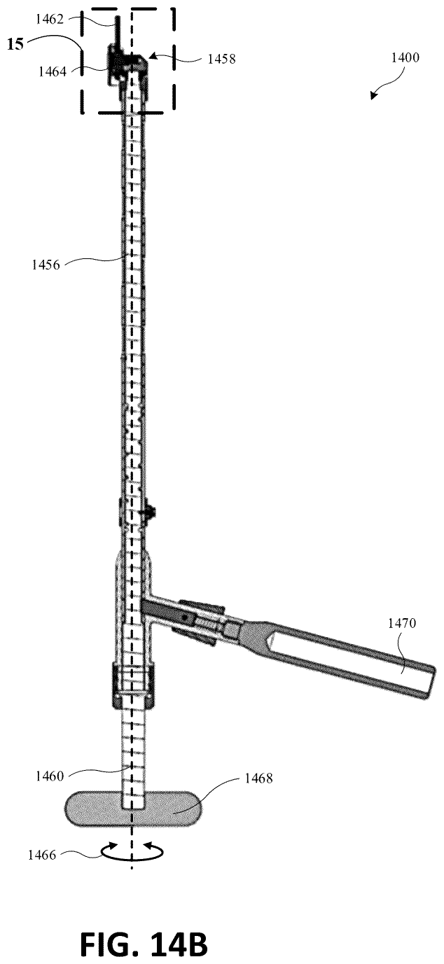

FIG. 14A illustrates a perspective view of a driver 1400 that is configured to engage the neck 1104 described above to adjust the length of the neck 1104. FIG. 14B is a cross-sectional view of the driver 1400 shown in FIG. 14A. As such, the driver 1400 in conjunction with the trial femoral assembly 1100 may be considered a trial femoral system.

The driver 1400 may include a driveshaft 1456 and a mating portion 1458 coupled to the driveshaft 1456. As discussed above, the driver 1400 may engage the center gear 1116 (e.g., the groove 1352 of the center gear 1116) and mesh with the teeth 1354 of the center gear 1116 to rotate the center gear 1116 about the longitudinal axis 1226 of the neck 1104 in the direction 1228. To facilitate such engagement, the driveshaft 1456 may be configured to rotate about a longitudinal axis 1460 of the driveshaft 1456 to drive the mating portion 1458 and thus drive the center gear 1116.

By way of example, the mating portion 1458 may include a fork 1462 coupled to a body 1464 of the mating portion 1458. The fork 1462 may be configured to engage the groove 1352 of the center gear 1116 to removably couple the fork 1462 to the neck 1104. Based on this engagement, the mating portion 1458 may engage the teeth 1354 of the center gear 1116. As a result, the mating portion 1458 may rotate the center gear 1116 about the longitudinal axis 1226 of the neck 1104 based upon rotation of the driveshaft 1456 about the longitudinal axis 1460 of the driveshaft 1456 in the direction 1466.

The driver 1400 may also include a handle 1468 that is coupled to the driveshaft 1456. For example, the handle 1468 may be a t-handle, a knob, a lever, a wheel, other mechanism, or a combination thereof that facilitates a user (e.g., the surgeon) interfacing with the driver 1400 to rotate the driveshaft 1456 about the longitudinal axis 1460 of the driveshaft 1456. However, it should be noted that the driveshaft 1456 may be pneumatically and/or electronically actuated. As such, manual driving of the driveshaft 1456 may not be needed.

The driver 1400 may also include one or more adjustment mechanisms to adjust one or more parameters of the driver 1400. For example, the driver 1400 may include the adjustment mechanism 1470. The adjustment mechanism 1470 may be configured to adjust tension on the driveshaft 1456. As a result, the adjustment mechanism 1470 may provide a safety mechanism to prevent movement of the driveshaft 1456 when the adjustment mechanism 1470 engages the driveshaft 1456. Similarly, the driver 1400 may also include an indicator (e.g., a slider, follower, display, etc.) that may display a readout to the user (e.g., the surgeon) to indicate a total amount of expansion of the neck 1104 that has been achieved.

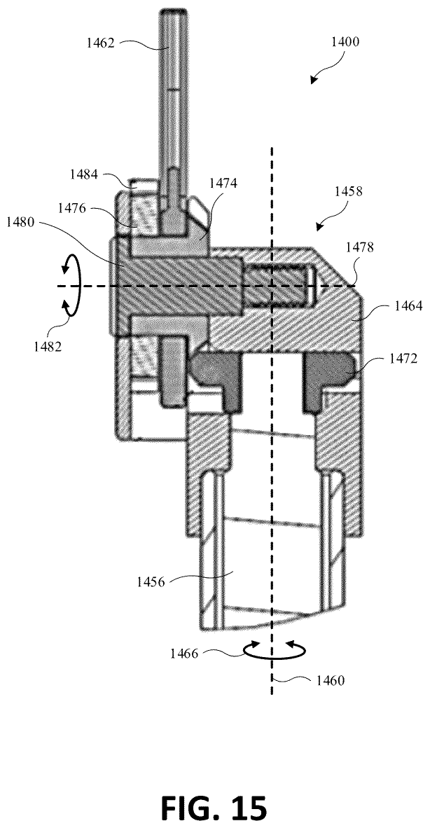

To provide further detail of the driver 1400, FIG. 15 illustrates close-up view 15 of the driver 1400 shown in the FIG. 14B. As discussed above, the driver 1400 may include the mating portion 1458 coupled to the driveshaft 1456 of the driver 1400. The mating portion 1458 may also include the fork 1462, which may be configured to engage the groove 1352 of the center gear 1116 to removably couple the driver 1400 to the neck 1104.

The mating portion 1458 may also include one or more gears. The one or more gears may be configured to transfer rotation of the driveshaft 1456 to the center gear 1116 to rotate the center gear 1116 about the longitudinal axis 1226 of the neck 1104. For example, the mating portion 1458 may include a first gear 1472, a second gear 1474, and a third gear 1476.

The first gear 1472 may be coupled to an end of the driveshaft 1456 and disposed in a portion of the body 1464 of the mating portion The first gear 1472 may rotate in the direction 1466 based upon rotation of the driveshaft 1456 in the direction 1466. The first gear 1472 and the driveshaft 1456 may be coaxial and rotate simultaneously with each other.

The second gear 1474 and the third gear 1476 may be coupled to, or otherwise in communication with, one another such that rotation of the second gear 1474 may drive rotation of the third gear 1476, or vice versa. The second gear 1474 and the third gear 1476 may be coaxial with one another about an axis of rotation 1478 of the second gear 1474 and the third gear 1476. The second gear 1474 and the third gear 1476 may also be rotatably coupled to the body 1464 of the mating portion 1458 via one or more fasteners, such as the fastener 1480.

To drive rotation of the center gear 1116, the driveshaft 1456 may be rotated in the direction 1466 about the longitudinal axis 1460 of the driveshaft 1456, thereby also rotating the first gear 1472 in the direction 1466. The first gear 1472 may be in communication with (e.g., engaged to) the second gear 1474 such that rotation of the first gear 1472 may drive rotation of the second gear 1474. By way of example, the second gear 1474 may be positioned substantially perpendicular to the first gear 1472 such that rotation of the second gear 1474 in the direction 1482 is substantially perpendicular to rotation of the first gear 1472 in the direction 1466. However, the axis of rotation 1478 of second gear 1474 may be nonparallel to the longitudinal axis 1460 of the driveshaft 1456 and form an angle other than substantially perpendicular.

As the second gear 1474 rotates in the direction 1482, the third gear 1476 also rotates in the direction 1482. As a result, teeth 1484 of the third gear 1476 may engage (e.g., mesh with) the teeth 1354 of the center gear 1116 to thereby rotate the center gear 1116 as described above. Thus, the driver 1400 via the first gear 1472, the second gear 1474, and the third gear 1476 may drive articulation of the neck 1104 to adjust the length of the neck 1104.

Based on the above, the advantage of this trial femoral assembly is an expandable trial neck that will reduce operating room time by allowing the surgeon to determine the appropriate trial implants (either head offset or stem offset) without dislocating the joint. The surgeon will be able to use an instrument that could provide tactile feedback by clicking at each offset size so that instead of dislocating the joint, replacing the head, and reducing the joint again, the surgeon will be able to leave the joint reduced and turn the instrument to change the offset size.

This technology will also reduce instrumentation significantly because there could be one trial neck for both standard and lateralized necks. There will also only need to be one neutral offset (+0 millimeter) head that the surgeon will trial with. Reducing instrumentation is especially important in joint arthroplasty due to the increasing presence of ambulatory surgery centers that do not have room to store large amounts of instrumentation. Having less instrumentation also makes things much easier for the scrub technician, making the whole procedure much smoother and quicker.

From the foregoing, it can be seen that the present disclosure accomplishes at least all of the stated objectives.

### LIST OF REFERENCE CHARACTERS

The following table of reference characters and descriptors are not exhaustive, nor limiting, and include reasonable equivalents. If possible, elements identified by a reference character below and/or those elements which are near ubiquitous within the art can replace or supplement any element identified by another reference character.

TABLE 1
List of Reference Characters

| | |
|---|---|
| 10 | Trial femoral head and neck assembly |
| 12 | Adjustable femoral neck portion |
| 14 | First length of an adjustable femoral neck portion |
| 16 | Expansion of neck portion along neck axis |
| 18 | Second length of an adjustable femoral neck portion |
| 20 | Trunnion |
| 22 | Stem or broach |
| 24 | Standard position of the stem or broach in relation to an adjustable |
| | femoral neck portion |
| 26 | Lateral change of stem or broach in relation to an adjustable |
| | femoral neck portion |
| 28 | Lateralized position of the stem or broach to an adjustable femoral |
| | neck portion |
| 50 | First embodiment of a neck-lengthening device |
| 52 | Tilted spiral cam |
| 54 | Housing |
| 56 | Neck portion |
| 58 | Protrusion |
| 59 | Opening |
| 60 | Notched rod |
| 62 | Longitudinal neck movement |
| 64 | Spiral cam movement that converts to longitudinal neck movement |
| 66 | Extension of the neck portion |
| 70 | Second embodiment of a neck-lengthening device |
| 72 | Driver tool |
| 74 | Enclosed rotating shaft |
| 75 | Direction of rotation of a rotating shaft |
| 78 | Bevel pinion gear |
| 80 | Bevel gear |
| 82 | Coupling sleeve |
| 84 | Connecting screws |
| 86 | Direction of rotation of the femoral head |
| 88 | Bevel pinion gear bushing |
| 89 | Circular protrusion |
| 90 | Threaded internal passage |
| 92 | Screw |
| 94 | Retained flange as an upper component of the coupling sleeve |
| 96 | Slot located within the trunnion for retaining the retained flange |
| 100 | Third embodiment of a neck-lengthening device |
| 102 | First nested, telescoping threaded element |
| 104 | Second nested, telescoping threaded element |
| 106 | Third outer nested, telescoping threaded element |
| 108 | Nut |
| 110 | Rotation of threaded elements |
| 112 | Direction upward and downward of the second nested, telescoping |
| | threaded element |
| 114 | Direction upward and downward of the third nested, telescoping |
| | threaded element |
| 116 | Anti-rotation guide |
| 1100 | Trial Femoral Assembly |
| 1102 | Broach |
| 1104 | Neck |
| 1106 | Head |
| 1108 | Ribs |
| 1110 | Distal End of the Broach |
| 1112 | Proximal End of the Broach |
| 1114 | Direction of Movement of the Neck |
| 1116 | Center Gear |
| 1218 | Base |
| 1220 | Projection of the Base |
| 1222 | Receiving Portion of the Base |
| 1224 | Trunnion |
| 1226 | Longitudinal Axis of the Neck |
| 1228 | Direction of Rotation of the Center Gear |
| 1230 | Pin |
| 1232 | Cavity of the Center Gear |
| 1234 | Cavity of the Base |
| 1236 | Cavity of the Trunnion |
| 1238 | Cylindrical Body of the Center Gear |
| 1240 | Direction of Translation of the Center Gear and/or Trunnion |
| 1242 | External Threading of the Center Gear |
| 1244 | Internal Threading of the Center Gear |
| 1246 | Threading of the Trunnion |
| 1248 | Threading of the Base |
| 1250 | Cylindrical Body of the Base |
| 1352 | Groove of the Center Gear |
| 1354 | Teeth of the Center Gear |
| 1400 | Driver |
| 1456 | Driveshaft |
| 1458 | Mating Portion of the Driver |
| 1460 | Longitudinal Axis of the Driver |
| 1462 | Fork |
| 1464 | Body of the Mating Portion |
| 1466 | Direction of Rotation of the Driveshaft |
| 1468 | Handle |
| 1470 | Adjustment Mechanism |
| 1472 | First Gear |
| 1474 | Second Gear |
| 1476 | Third Gear |
| 1478 | Axis of Rotation of the Second Gear and Third Gear |
| 1480 | Fastener |
| 1482 | Direction of Rotation of the Second Gear and Third Gear |
| 1484 | Teeth of the Third Gear |

### GLOSSARY

Unless defined otherwise, all technical and scientific terms used above have the same meaning as commonly understood by one of ordinary skill in the art to which embodiments of the present disclosure pertain.

The terms “a,” “an,” and “the” include both singular and plural referents.

The term “or” is synonymous with “and/or” and means any one member or combination of members of a particular list.

As used herein, the term “exemplary” refers to an example, an instance, or an illustration, and does not indicate a most preferred embodiment unless otherwise stated.

The term “about” as used herein refers to slight variations in numerical quantities with respect to any quantifiable variable. Inadvertent error can occur, for example, through use of typical measuring techniques or equipment or from differences in the manufacture, source, or purity of components.

The term “substantially” refers to a great or significant extent. “Substantially” can thus refer to a plurality, majority, and/or a supermajority of said quantifiable variables, given proper context.

The term “generally” encompasses both “about” and “substantially.”

The term “configured” describes structure capable of performing a task or adopting a particular configuration. The term “configured” can be used interchangeably with other similar phrases, such as constructed, arranged, adapted, manufactured, and the like.

Terms characterizing sequential order, a position, and/or an orientation are not limiting and are only referenced according to the views presented.

The “invention” is not intended to refer to any single embodiment of the particular invention but encompass all possible embodiments as described in the specification and the claims. The “scope” of the present disclosure is defined by the appended claims, along with the full scope of equivalents to which such claims are entitled. The scope of the disclosure is further qualified as including any possible modification to any of the aspects and/or embodiments disclosed herein which would result in other embodiments, combinations, sub-combinations, or the like that would be obvious to those skilled in the art.

What is claimed is:

1. A trial femoral assembly, comprising:
a broach configured to couple to a femoral shaft;
a neck removably coupled to the broach and that includes a center gear configured to rotate about a longitudinal axis of the neck to adjust a length of the neck; and
a head removably coupled to the neck and configured for insertion into a hip joint.

2. The trial femoral assembly of claim 1, wherein the center gear is configured to rotate about the longitudinal axis of the neck to adjust the length of the neck such that a distance between the broach and the head is increased or decreased.

3. The trial femoral assembly of claim 1, wherein the neck further includes:
a base configured to removably couple to the broach; and
a trunnion configured to removably couple to the head, wherein the center gear is located between the base and the trunnion.

4. The trial femoral assembly of claim 3, wherein the trunnion defines a cavity therein and a portion of the center gear is located within the cavity of the trunnion.

5. The trial femoral assembly of claim 4, wherein the trunnion includes threading located in the cavity of the trunnion configured to engage external threading of the center gear such that rotation of the center gear about the longitudinal axis translates the trunnion along the longitudinal axis to adjust the length of the neck.

6. The trial femoral assembly of claim 3, wherein the neck further includes a pin that is fixedly coupled to the trunnion and extends into a cavity of the base to maintain a rotational position between the trunnion and the base with respect to the longitudinal axis.

7. The trial femoral assembly of claim 6, wherein the center gear includes internal threading located within the cavity of the center gear that engages threading of the base such that rotation of the center gear about the longitudinal axis translates the center gear along the longitudinal axis to adjust the length of the neck.

8. A neck of a trial femoral assembly, comprising:
a base configured to removably couple to a broach of the trial femoral assembly and that defines a base cavity therein;
a trunnion configured to removably couple to a head of the trial femoral assembly and that defines a trunnion cavity therein;
a pin fixedly coupled to the trunnion; and
a center gear that defines a center gear cavity therein, wherein the pin is configured to extend into the center gear cavity and into the base cavity, and the center gear is configured to rotate about a longitudinal axis of the neck to translate at least one of the center gear and the trunnion along the longitudinal axis.

9. The neck of claim 8, wherein rotation of the center gear about the longitudinal axis increases or decreases a distance between the trunnion and the base.

10. The neck of claim 8, wherein the center gear includes:
external threading configured to engage threading of the trunnion;
internal threading configured to engage threading of the base; and
teeth, wherein a driver is configured to engage the teeth to rotate the center gear about the longitudinal axis.

11. The neck of claim 10, wherein the center gear further includes a groove located adjacent to the teeth and that is configured to engage the driver.

12. The neck of claim 10, wherein the threading of the trunnion is located within the trunnion cavity and the internal threading of the center gear is located within the center gear cavity.

13. The neck of claim 8, wherein the pin extends along the longitudinal axis.

14. The neck of claim 13, wherein the pin, the base, and the center gear are coaxial along the longitudinal axis.

15. The neck of claim 14, wherein the center gear is positioned between the trunnion and the base along the longitudinal axis.

16. The neck of claim 8, wherein both the center gear and the trunnion are configured to translate along the longitudinal axis based upon rotation of the center gear about the longitudinal axis.

17. A trial femoral system, comprising:
a trial femoral assembly that includes:
a broach configured to be coupled to a femoral shaft;
a neck removably coupled to the broach and that includes a center gear configured to rotate about a longitudinal axis of the neck to adjust a length of the
neck; and
a head removably coupled to the neck; and
a driver configured to engage the neck to adjust the length of the neck, wherein the driver includes:
a driveshaft configured to rotate about a longitudinal axis of the driveshaft; and
a mating portion configured to engage the center gear and rotate the center gear about the longitudinal axis of the neck based upon rotation of the driveshaft about the longitudinal axis of the driveshaft.

18. The trial femoral system of claim 17, wherein the mating portion includes:
a fork that is configured to engage a groove of the center gear to removably couple the driver to the neck; and
a gear comprising teeth that are configured to engage teeth of the center gear.

19. The trial femoral system of claim 18, wherein rotation of the driveshaft about the longitudinal axis of the driveshaft rotates the gear about an axis of rotation that is nonparallel to the longitudinal axis of the driveshaft to thereby rotate the center gear about the longitudinal axis of the neck.

20. The trial femoral system of claim 19, wherein the axis of rotation of the gear is perpendicular to the longitudinal axis of the driveshaft.
